(19) United States
(12) Patent Application Publication
(10) Pub. No.: US 2016/0317131 A1
(43) Pub. Date: Nov. 3, 2016

(54) Medical diagnostic imaging ultrasound probe battery pack radio

(71) Applicant: Siemens Medical Solutions USA, Inc., Malvern, PA (US)

(72) Inventors: Jodi Schwartz Klessel, North Wales, PA (US); Joseph Urbano, Audubon, PA (US); George Ku, East Norriton, PA (US)

(21) Appl. No.: 14/699,568

(22) Filed: Apr. 29, 2015

(51) Int. Cl. A61B 8/00

(52) U.S. Cl. CPC A61B 8/56; A61B 8/4472

(57) ABSTRACT

In an ultrasound imaging system, a wireless radio is included as part of a removable battery pack. The charge, signals used for locating the battery, and other information may be wirelessly communicated from the battery pack even when not connected with an ultrasound transducer probe. Queries, configuration data and other information may be communicated from the ultrasound system or locator device to the probe battery and its circuitry. The same radio may be used by the ultrasound transducer probe when connected. Alternatively, a different radio is used by the probe.

### BACKGROUND

This present embodiments relate to ultrasound diagnostic imaging. In particular, the embodiments relate to wireless ultrasound probes used in ultrasound imaging.

For scanning and imaging, a radio in the wireless probe communicates with the imaging system. Wireless ultrasound probes have the potential to get lost due to their small size and lack of cable connection. This is especially true in sterile procedures, where it is possible to discard a probe along with disposable items at the end of the procedure. To avoid loss of the probe, the received signal strength from the probe being below a threshold may be used as an indication that the probe is separated from the system by a large distance. This separation triggers an audible alert from the probe.

Wireless probes may have removable battery packs, which may be lost when separated from the probe. For ease of use, the battery packs are to be maintained with sufficient charge. The battery charge status is read by the system when the battery is mated to the probe or when the battery pack is connected to one of the imaging system's charger bays. While adherence to a battery maintenance process may avoid situations where no charged battery is available for imaging, human error may result in batteries being not adequately charged prior to use.

### BRIEF SUMMARY

By way of introduction, the preferred embodiments described below include methods, systems, and transducer probes for communicating in ultrasound imaging. A wireless radio is including as part of a removable battery pack. The charge, signals used for locating the battery, and other information may be wirelessly communicated from the battery pack even when not connected with an ultrasound transducer probe. Queries, configuration data and other information may be communicated from the ultrasound system or locator device to the probe battery and its circuitry. The same radio may be used by the ultrasound transducer probe when connected. Alternatively, a different radio is used by the probe.

In a first aspect, a system is provided for communications with an ultrasound scanner. An ultrasound transducer probe includes a probe housing and a transducer array in the probe housing. A battery pack includes a battery pack housing. The battery pack is configured for removable mating with the transducer probe. A wireless radio is in the battery pack housing. The wireless radio is configured for wireless communications with a remote device.

In a second aspect, an ultrasound system is provided for communications. A battery enclosure encloses a battery. A cableless ultrasound transducer probe is releasably connectable with the battery enclosure. A transceiver is within or on the battery enclosure. An ultrasound imager is configured to generate an ultrasound image from data received from the cableless ultrasound transducer probe.

In a third aspect, a method is provided for communicating in an ultrasound system. A removable battery pack mates with an ultrasound transducer probe. A radio in the battery pack wirelessly transmits information. A remote device receives the information.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of an ultrasound system with wireless communications;

FIG. 2 is a block diagram of one embodiment of an ultrasound system using wireless communication; and

FIG. 3 is a flow chart diagram of one embodiment of a method for communicating for an ultrasound system.

### DETAILED DESCRIPTION OF the DRAWINGS AND PRESENTLY PREFERRED EMBODIMENTS

A radio is located within a removable battery pack of an ultrasound probe. The radio located within the battery pack may communicate battery charge levels when the pack is not mated to the probe. For wireless ultrasound probes with removable battery packs, the process of managing the charge state of battery packs is improved. Using a cost-effective low-power radio, a locator function for lost probes and/or battery packs may be provided. An active radio-frequency locator system may use transmissions from the battery pack. For wireless ultrasound probes that have replaceable battery packs but no locator function, providing the radio in the battery pack may provide a low-cost upgrade. Similarly, as new radio standards, techniques, or systems are developed, the radio communications from the wireless probe may be more cost-effectively upgraded by merely replacing the battery pack rather than the entire probe.

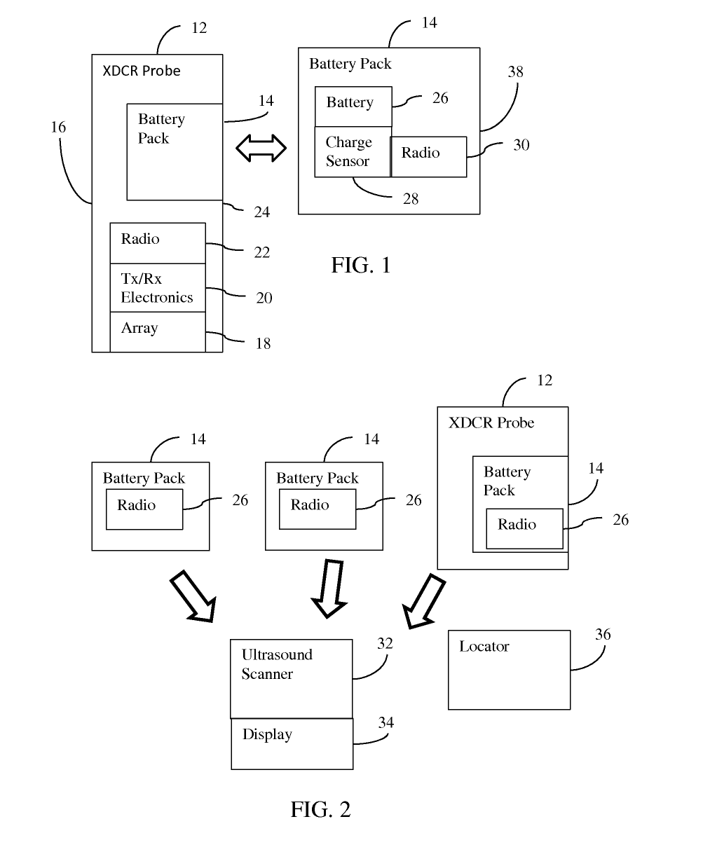

FIG. 1 shows one embodiment of an ultrasound system with wireless communications. The system includes a wireless transducer probe 12 and a removable battery pack 14. The communications occur while connected to the probe 12 (left side of FIG. 1) or while disconnected from the probe 12 (right side of FIG. 1). The battery pack 14 shown disconnected is enlarged relative to the battery pack 14 shown connected to the probe 12. The communications are from the battery pack 14 to another device, such as a remote device. Remote is used to indicate a device not in direct physical contact or not connected through a conductor. The remote device may or may not be in a same room as the battery pack 14. In one embodiment, the remote device is a locator for locating the battery pack 14.

The ultrasound transducer probe 12 includes a probe housing 16, an array 18, transmit and/or receive electronics 20, and a radio 22. The ultrasound transducer probe 12 is wireless, so a cable does not connect the probe 12 to an ultrasound imager. Instead, ultrasound data generated by scanning a patient with the array 18 and the transmit and/or receive electronics 20 is wirelessly transmitted to an ultrasound imager. The ultrasound imager generates an ultrasound image representing the patient from the data wirelessly received from the probe 12.

Additional, different, or fewer components may be provided. For example, the radio 22 is not provided. Instead, the radio 30 of the battery pack 14 is used for probe 12 communications. As another example, other ultrasound imaging processing circuits, detectors, or hardware is provided in the probe 12.

The probe housing 16 is plastic, fiberglass, metal, rubber, epoxy, resin, other material, or combinations thereof. For example, different types of plastic are used, one for an acoustic window adjacent to the array 18 and another as a liquid and physical protector. A rubber or other grip material may be added to an outside or integrated as part of the probe housing 16 for ergonomic comfort or control during use. A single piece or multiple pieces form the probe housing 16. For multiple pieces, snap fit, magnets, screws, glue, epoxy, nuts and bolts, or other now known or later developed connections between different pieces are provided.

Buttons, sliders, or other input devices may be included on or in the probe housing 16. For example, a touch sensitive (e.g., capacitive sensor) is provided to determine that the probe housing 16 is being held. As another example, a gyroscope or accelerometer may be provided to sense movement of the probe housing 16. Alternatively, no input devices are provided. Output devices, such as a display screen, light emitting diode, or speaker may be provided. For example, a light emitting diode or diodes are provided for indicating selection of the probe 12, power on/off, and/or status information. In other embodiments, no output device is provided.

The probe housing 16 is shaped and sized for being held by a hand of a user. The probe housing 10 is for handheld use external to the patient. The sonographer grips or holds the probe housing 16 for scanning a patient along the surface of the skin. In alternative embodiments, the probe housing 16 is a part of an endocavity, intraoperative or other transducer housing with a portion of the housing used internally of the patient. The ergonomic aspects of the transducer housing are shaped for user gripping within the patient (e.g., intraoperative probe) or for holding a portion of the probe that is external to the patient (e.g., transesophageal probe).

The probe housing 16 includes a surface sized as appropriate for a user's hand. For example, the surface has a width a little less than, at or a little wider than the palm, allowing the thumb and fingers to grip at least partially around the probe housing 16. A finger grip may be provided as part of the probe housing 16. The finger grip is a shaped area, such as an indentation, ridges, bumps, dimples, crevices, or other structure for accepting the fingers and/or thumb of the user. In one embodiment, the finger grip is formed of the same material as the probe housing 16. In other embodiments, other materials form the finger grip, such as an elastomer pad bound to the probe housing 16. The elastomer pad may extend beyond the finger grip. The exterior of the probe housing 16 forms an ergonomic shape. The user's grip fits naturally over the probe housing 16.

The probe housing 16 is of any size, but may be less than ten inches along a longest dimension in one embodiment. By having a smaller size or volume, the probe 12 may more easily be manipulated by the sonographer to scan a patient.

The probe housing 16 is free of a cable. Rather than using a cable to communicate control information and/or scan data to and/or from the probe 12, one of the radios 22, 30 is used. By providing a wireless radio, the probe 12 and corresponding probe housing 16 may be free of cable connection.

The probe housing 16 includes a connector for releasably connecting with the battery pack 14. Slots, latches, snap fit, pressure fit, screws, bolts, combinations thereof, or other physical connectors may be used. The probe housing 16 includes a part for physically mating with part of the battery pack 14, so the mating parts have a same or compatible surface contour. Metal fingers, springs, buttons, contact pads, or other structures may be provided for electrical connection. When or as the physical connection occurs, electrical connection is established. In addition or alternatively, inductive coupling may be used for communicating control information between the battery pack 14 and probe 12 or across the probe housing 16.

In one embodiment, the probe housing 16 includes an indentation 24. The indentation 24 is sized to accept the entire battery pack 14. The depth of the indentation 24 may correspond to or be similar to a depth of the battery pack 14 so that one surface of the battery pack 14 is adjacent a surrounding surface of the probe housing 16 to form a continuous or smooth outer surface. In another embodiment, the battery pack 14 slides into the indentation 24 and a lid or cover is placed over the indentation 24 to cover the inserted battery pack 14. In alternative embodiments, the battery pack 14 as mated or connected extends beyond or sticks out from the probe housing 16.

The connection is releasable. The cableless ultrasound transducer probe 12 releasably connects with the battery enclosure or housing 38 of the battery pack 14. A latch, snap, pressure, release, or other mechanism allows for the battery pack 14 to be removed from the probe housing 12 so that no physical or electrical direct connection remains. Due to the releasable connection, a different battery pack 14, such as one with a charge, may be connected to the probe 12.

The array 18 is a transducer array of elements. The elements are in a one or two-dimensional array, such as being a one-dimensional linear or curved array of 16-256 elements. Alternatively, an array with fewer elements, such as an annular array with 2-16 elements, may be mechanically swept over the image field of view. Any now known or later developed transducer array may be used. The elements are piezoelectric or capacitive membrane elements for transducing between electrical and acoustic energy. The array 18 is positioned by, in, or on the probe housing 16 to allow acoustic scanning of the patient.

The transmit and/or receive electronics 20 are amplifiers, filters, pulsers, transmit/receive switches, transmit beamformer, receive beamformer, multiplexer, analog-to-digital converters, or other now known or later developed electronics for operating the probe 12. For ultrasound scanning, relatively delayed and/or phased electrical waveforms are applied to the array 18 to form transmit beams or virtual point sources. Acoustic transmissions from single array elements may also serve as point sources. Electrical signals generated by the array 18 in response to the acoustic transmission are amplified and delayed and/or phase adjusted to form receive beams or focused pixels. The receive beams or focused pixels are detected (e.g., B-mode or intensity detection or flow estimation), scan converted, and mapped to display values. The transmit and/or receive electronics 20 may perform all or only part of this ultrasound image processing. In one embodiment, the transmit and/or receive electronics 20 include pulsers for generating relatively delayed and/or phased electrical waveforms for transmit operation and include amplifiers and analog-to-digital converters for wireless transmission of element data to a remote device, which performs receive beamforming or pixelforming and other ultrasound imaging functions. In another embodiment, the transmit and/or receive electronics 20 include a receive beamformer for full or partial receive beamforming. A multiplexer may be provided for combining data to reduce wireless bandwidth requirements for transmitting the ultrasound data.

The transmit and/or receive electronics 20 include analog and/or digital circuits. One or more chips, such as application specific integrated circuits or field programmable gate arrays, may be included. Any electronics for transmit and/or receive operations may be used.

The transmit and/or receive electronics 20 are within the probe housing 16. The probe housing 16 encloses or partially encloses the electronics 20. The electronics 20 connect with the array 18, radio 22, and/or radio 30 through wires, traces, or other conductors.

The radio 22 is a transceiver, transmitter, or receiver. The radio 22 includes an antenna and circuits for transmitting and/or receiving signals. In one embodiment, the radio 22 is a Bluetooth radio. The radio 22 may also be a WiFi, UWB, or any other radio type. A chip, application specific integrated circuit, and/or other device may implement the processing of the radio 22 as well as provide an integrated antenna.

The radio 22 is in the probe housing 16, but outside the battery pack 14. The probe housing 16 encloses or at least partially encloses the radio 22. Power for the radio 22 and electronics 20 is provided by electrical connection with the battery pack 14.

The radio 22 is configured to transmit ultrasound data from the probe 12 in a radio frequency format. The received signals, such as digital samples of the element signals, beamformed samples representing locations in the patient, or detected data are transmitted by the radio 22 to a remote device for further ultrasound processing and generation of an ultrasound image. The ultrasound data represents acoustic response of the patient acquired using the array 18 to acoustically scan the patient. The ultrasound data transmitted by the radio 22 is electrical signal or digital data after processing by the electronics 20. The radio 22 provides for wireless or cableless operation of the probe 12 for ultrasound imaging of the patient by the remote device.

The radio 22 may receive control information from the remote device. The operation of the probe 12, such as the scan pattern or transmit waveform characteristics, is controlled by the control information. In alternative embodiments, the control information is stored on the probe 12 and not received through the radio 22. Other control information, such as battery status or probe temperature, may be sent from probe 12 using radio 22. Alternatively, this control information is generated on the probe 12 and not sent through radio 22.

In one embodiment, the radio 22 is provided for transmitting acquired ultrasound data, but not for probe location or may use high power and/or bandwidth signals for probe location, resulting in more rapid battery drain. By providing a radio 30 in the battery pack, lower power radio operation may provide for less drain in locating the battery pack 14 and connected probe 12. An existing probe 12 with the radio 22 may benefit from addition of the radio 30 in the battery pack 14. In other embodiments, the radio 30 in the battery pack 14 is the only radio and radio 22 is not provided. The radio 30 in the battery pack 14 receives scan instructions and transmits status information and ultrasound data for the probe 12. In yet another embodiment, the radio 30 in the battery pack 14 is used for transmitting and receiving control information, while the radio 22 is used for transmitting ultrasound data for the probe 12.

When the battery pack 14 is disconnected from the probe 12, the radio 22 may not provide ultrasound data or other signals to a remote device. While an additional battery may be included within the probe housing 16 to allow for location signals, the addition of a battery may incur additional cost and complexity. The radio 22 may communicate battery status for any connected battery pack 14, but not of disconnected battery packs 14. Since a probe 12 is most likely to be lost shortly after a procedure, when a battery pack 14 is mated with the probe 12, locating the battery pack 14 will also locate the probe 12.

The battery pack 14 includes one or more batteries 26, a charge sensor 28, a radio 30, and a battery pack housing 38. Additional, different, or fewer components may be provided. For example, a processor or other controller of power, sensing, and/or the radio 30 is included. As another example, a speaker, light emitting diode, or other visual and/or audio output device is included in or on the battery pack 14. In yet another example, the charge level sensor 28 is not provided.

The battery 26 is any now known or later developed bundle of one or more batteries. One or more nickel cadmium (NiCad), nickel metal hydride (NiMH), lithium ion (Li Ion), lithium polymer (Li-Po), sealed lead acid, or other batteries connected together for powering the radio 22, electronics 20, radio 30, sensor 28, and/or other devices. The battery pack 14 is rechargeable. In alternative embodiments, the battery pack 14 provides an initial charge but is not rechargeable.

The battery pack housing 38 is plastic, rubber, resin, epoxy, or other electrically insulating material. The battery pack housing 38 encloses the batteries 26. One or more electrical contacts may be exposed on or through a hole in the battery pack housing 38. Electrical signals may alternatively or additionally be inductively coupled across battery pack housing 38. The batteries 26 are enclosed, at least partially, by the battery pack housing 38.

The battery pack housing 38 is configured for removable mating with the transducer probe 12. In one embodiment, the battery pack housing 38 has a three-dimensional orthotope or rectangular prism shape, but other shapes may be used. One part of the battery pack housing 38 includes a surface with a contour matching or conforming to part of the probe housing 16. The size similarly matches for mating the battery pack 14 in or on the probe 12. Latches, grooves, magnets, holes, or other devices may be provided for physical and/or electrical mating with the probe 12.

When mated with the transducer probe 12, the battery pack 14 powers the transmit and receive electronics 20, the radio 22, and/or any other electronics of the probe 12. The circuits of the cableless ultrasound transducer probe 12 are powered by the batteries 26 when the battery housing 38 is connected with the probe 12.

The charge level sensor 28 is a circuit, chip, processor, or other device for sensing a charge of the batteries 26. In one embodiment, the charge level sensor 28 is part of a circuit or processor for controlling power output and/or charging of the batteries 26. The charge level sensor 28 connects with the radio 30 for outputting a charge level or other measure of battery performance or status.

The wireless radio 30 is the same or different type of transceiver, receiver, or transmitter as the radio 22. In one embodiment, the wireless radio 30 is an application specific integrated circuit (chip) implementing a low-power radio, such as a radio operating pursuant to the Bluetooth low energy standard. Any low power radio that can operate at a low power consumption such as 0.01 to 0.5 mW for an extended period, may be used. In another embodiment, the radio 30 may operate in either low power or higher power modes. For example, when using signals from the radio 30 for locating or non-ultrasound scanning operation, low power transmissions at a pre-determined power or amplitude level are used. When transmitting ultrasound data, high or higher power or amplitude level transmissions are used. Alternatively, the radio 30 operates just at a high power level, such as not being configured for the Bluetooth low energy operation, or just at the low power level. In one embodiment, the radio 30 enters a very low power sleep mode, which is interrupted every 2 seconds for brief radio activity, such that the average power is approximately 0.03 mW, and at that average power level, charge is maintained on batteries 26 for six months after an initial charge of only 5% of total capacity. In such an embodiment, a reserve charge of approximately 5% may be preserved on batteries 26 during normal scanning operation, so that the radio 30 may be operated in a low-power mode over an extended period of time (e.g., six months) when probe 12 is not used for scanning.

The radio 30 is in the battery pack housing 38. The chip, circuit, or other device or devices embodying the radio 30 are enclosed within the battery pack housing 38. An antenna may be integrated on an outside of the battery pack housing 38 or is enclosed within the housing 38. Since the radio 30 is in the battery pack 14, the batteries 26 power the radio 30.

Including the radio 30 in the battery pack 14 may allow for lower cost upgrading of radio or radio function of the probe 12. Instead of replacing the probe 12, just the battery pack 14 is replaced to provide an improved radio 30. The replacement may provide an upgraded radio, such as a radio with more energy efficient operation, greater bandwidth, or other performance increase. Since battery packs 14 may have a shorter lifetime and lower cost than the probe 12, upgrading of the radio 30 may be more cost effective.

Providing the radio 30 in the battery pack 14 may avoid the need for the radio 22 in the probe 12, or allow the radio 22 to have reduced capability, such as uni-directional operation instead of bi-directional. Mated probe 12 may be provided with a simpler radio 22, or only a single radio 30, the one in the battery pack 14, may be provided for the mated probe 12 and battery pack 14. In this case, the radio 30 residing in the battery pack 14 is used for normal probe-to-system communication.

The radio 30 is configured for wireless communications with a remote device. The remote device may be a base unit or ultrasound imager. Alternatively or additionally, the remote device is a personal computer, tablet, or smartphone.

For wireless communication, the radio 30 transmits a radio-frequency signal. Any type of signal may be transmitted, such as a pulse signal, an identification signal, or a network connection signal. The battery pack radio transmission occurs either at a periodic rate or in response to a signal transmitted by the remote device (e.g., the radio 30 receives a request to transmit from a locator device or ultrasound imager).

In one embodiment, the radio 30 is configured to transmit a charge level of the battery pack 14. The charge level or other status information provided by the charge level sensor 28 is transmitted. The level of the charge or a value derived from the level of the charge is output. For example, if the charge falls below a given level (e.g., 15%), a signal is output by the radio 30. The signal is the charge level or is a warning signal not indicating a specific charge.

Since the radio 30 is in the battery pack 14, the charge level may be transmitted when the battery pack 14 is not mated with the probe 12. When mated, the radio 30 may also transmit the charge level. The charge level is transmitted regardless of whether the battery pack 14 is connected with the probe 12. Alternatively, the charge level is only transmitted or different information for charge level is transmitted depending on whether the battery pack 14 is mated or connected with the probe 12.

In one embodiment, a battery charge reserve is maintained to allow the radio 30 to stay powered for an extended period, such as for days, weeks, or months. Any reserve may be used, such as 5%. If mated to a probe 12, all or some other probe functions are disabled if the charge state is below the reserve threshold. The location and/or charge status functions are allowed to occur.

Another signal may be for radio-frequency location operation. A locator signal is transmitted. The locator signal may be a specific signal for location or may be a signal used for other purposes, such as a radio identifier signal. The locator signal is transmitted at a predetermined amplitude where the location determination is based on signal strength. Variable or other non-predetermined amplitudes may be used, such as where triangulation is used.

The locator signal is used to locate the probe in one or more ways. In one approach, the locator signal indicates a distance from a locator device to the probe. The signal strength of the transmission as received at the locator device indicates the distance. By moving the locator device, an indication may be output of increasing or decreasing signal strength, allowing a user to find the probe 12 or battery pack 14. The distance may additionally or alternatively be output to the user. The signal strength may be mapped to the distance.

In another approach, the locator signal is used to indicate a change in location. If the signal strength changes or falls below a threshold, then the battery pack 14 is indicated as moving too far away from the locator device. The locator device transmits a control signal to the radio 30. The radio 30 causes the battery pack 14 or connected probe 12 to emit a visual or audio output for locating the battery pack 14 or connected probe 12. The locator or ultrasound imager may command the battery pack 14, via the radio 30, to flash a light or beep its beeper. The light and/or audio may be coded or different for different information. For example, the light or audio indicates that the battery 26 is the one in the vicinity with the greatest charge level. Different light and/or audio indicates that the charge level is below a threshold. Yet other light and/or audio sequence indicates that the battery pack 14 is being moved away from the locator device.

The radio 30 is used for recovering lost probes 12 mated with the battery pack 14. Battery packs 14 or connected probes 12 may be prevented from being lost by monitoring signal strength. Other location approaches may be used. For example, other location sensing than signal strength is used (e.g., ultrasound). The radio 30 transmits and/or receives signals for coordinating response to the detected location and/or for detection of the location.

The radio 30 may be configured to transmit ultrasound data from the probe 12. When the battery pack 14 is connected to the probe 12, the radio 30 electrically connects with the transmit and/or receive electronics 20 of the probe 12. Data from the electronics 20 is provided to the radio 30 for transmission to a remote ultrasound imager. The element, beamformed or other data representing the scanned patient is wirelessly transmitted by the radio 30 for imaging or quantification.

Putting a low-power radio 30 within the removable battery pack 14 of the ultrasound probe 12 may enable the probes 12 in the field to be upgraded with newer capability. For example, a wireless probe 12 may be updated with a probe locator function. The probe 12 may include a radio 22, but the radio is not configured for the function. For example, the locator device is a smartphone or tablet operating pursuant to a particular standard for wireless communications. The added radio 30 of the battery pack 14 operates pursuant to the standard while the existing radio 22 does not. An application on the locator device allows for the location function using the radio 30 of the battery pack 14. Using the tablet or smartphone as the locator device may avoid having to upgrade the ultrasound imager for this function.

Where the ultrasound imager does not include a battery charger or other port for direct connection of the probe 12 to the ultrasound imager (e.g., standalone charger is used), the charge status of the battery may be unknown if not communicated to the radio 20 of the probe 12. By including the radio 30 in the battery pack 14, the charge status may be provided despite the lack of direct connection. The charge status of many batteries or a given battery is gathered, even if the battery or batteries are not in chargers or not connected to probes 12.

In one embodiment, the battery pack 14 or probe 12 includes a sensor to detect mating of the battery pack 14 and the probe 12. The sensor is electrical, magnetic, or mechanical. The radio 30 of the battery pack 14 communicates the probe-mating status (i.e., whether connected) to the remote device. The probe-mating status may be used to determine which of the battery packs 14 are mated to which of the probes 12, which probes 12 are not mated, and/or which battery packs 14 are not mated.

Queries, configuration data and other information may be communicated from the ultrasound system or locator device to the radio 30 and the probe 12. For example, the battery radio 30 receives control data to re-configure the radio 30 within the battery pack 14 and/or to re-configure or update a microcontroller within the battery pack 14 or probe 12. Such configuration needs a higher bandwidth than provided by low power Bluetooth, so the battery pack radio 30 is reconfigured by the received control instruction to operate using regular Bluetooth or other greater bandwidth operation. The control instructions for reconfiguring the microcontroller are then received by the radio 30 and used to reconfigure.

It can often be very difficult to get radio approvals in multiple countries as each country has its own regulations. It may be advantageous to provide the radios 30 inside the removable battery pack 14, so that different packs may be designed and approved for various countries. For example, once a radio 30 has been approved in a certain subset of countries, its design cannot change without requiring recertification within those countries. However, getting approvals in additional countries may create the need for design changes to the radio 30, potentially creating a production configuration problem if the radio(s) 22 are embedded in the probe 12. Providing the radio(s) 30 in the battery pack results in redesign or change in the battery pack 14, not the more-expensive probe 12. Production then simply ships the country-specific battery packs 14 with a common probe 12 to each country. The various configurations may involve different frequency bands, bandwidths, modulation schemes, detect-and-avoid controls, power levels, or simply be design changes to reduce spurious emissions.

The probe 12 and battery pack 14 or packs 14 are used together with other devices. FIG. 2 shows one embodiment of a system for communications with an ultrasound scanner or imager 32. The probe 12 uses the radio 22 or 30 to communicate with the ultrasound imager 32 and/or locator 36. Spare battery packs 14 not connected with the probe 12 may be available and may likewise communicate.

Additional, different, or fewer components may be provided. For example, more or fewer battery packs 14 and/or ultrasound transducer probes 12 are provided. As another example, no locator 36 or additional locator devices 36 are provided. In yet another example, more than one ultrasound imager 32 and additional probes 12 are provided. The probes 12 pair or reversibly pair with imagers 32.

The battery packs 14 include radios 30 and batteries 26 in battery pack housings 38. In the example of FIG. 2, there are two spare battery packs 14 and one battery pack 14 connected with the probe 12. The spare battery packs 14 may be connected with other probes 12 or may be unconnected. The spare battery packs 14 may or may not be in charging stations.

The spare battery packs 14 are in a specific or known location, such as stacked by the imager 32, on a desk, in a drawer, or other location. Alternatively, the spare battery packs 14 are placed in unknown locations. Similarly, the probe 12 is in a known location (e.g., charging on a charging station of the imager 32) or placed in an unknown location. The battery packs 14 are remote from the imager 32 or may be connected to a charging station of the imager 32.

The radios 30 of the battery packs 14 transmit to and/or receive from the imager 32 and/or locator 36. The communications may be broadcast for any receiver or are addressed or coded for specifically paired devices. The information received by the radios 30 may be control signals, such as to activate a speaker or light or to configure a probe 12 for ultrasound scanning. For the radio 30 in the battery pack 14 mated to the probe 12, the information transmitted by the radio 30 may be ultrasound data derived from scanning a patient. For any of the radios 30 (i.e., in mated or unmated battery packs), the information transmitted may be network data (e.g., identification, pairing requests, or other data), heartbeat data, location signals, battery or charge status, or other data. Any of the types of information may be used for location functions.

The ultrasound imager 32 includes a display 36. The ultrasound imager 32 is a medical diagnostic ultrasound scanner, a computer, a server, or other device for generating and displaying ultrasound images of the patient on the display 36. The ultrasound imager 32 includes a receive beamformer, filter, detector, Doppler estimator, scan converter, memory, display plane, or combinations thereof. Additional, different, or fewer components may be provided.

The ultrasound imager 32 wirelessly receives the ultrasound scan data from the probe 12 and completes the ultrasound imaging process. Where the probe transmits element data, the ultrasound imager 32 receive beamforms, detects (B-mode, color flow mode, M-mode, pulse wave Doppler mode, harmonic mode, contrast agent mode, other mode, or combination thereof), spatially filters, temporally filters, scan converts, display maps, and outputs a resulting image. Additional, different, or fewer functions may be performed by the imager 32. For example, the probe 12 outputs receive beamformed data. The imager 32 does not perform the receive beamformation, so may or may not have a receive beamformer. As another example, the probe 12 outputs scan converted and/or display mapped data (e.g., outputs an ultrasound image for display), so the imager 32 provides the image on the display 36. The imager 32 is configured to generate an ultrasound image from data received from the cableless ultrasound transducer probe 12.

The display 34 is a CRT, liquid crystal diode, light emitting diode, plasma, printer, projector, or other display. In response to display values output by the imager 32, the display 34 presents an ultrasound image. A sequence of images from on-going scanning may be output.

The display 34 may also be used for user interface functions. Inputs or controls for configuring the ultrasound imager 32 and/or probe 12 are received from the user. The display 34 displays input options, confirms selections, and/or otherwise includes user interface feedback to the user. Any configuration information may be displayed. Information related to the probe 12 and/or battery packs 14 may be displayed, such as displaying pairing, location, battery status (e.g., charge level), or other information.

In one embodiment, the ultrasound imager 32 is configured to output location information. The display 34 or a speaker outputs a warning when a battery pack 14 with or without a connected probe 12 is moving away from or has passed a threshold distance (e.g., signal strength below a threshold). The ultrasound imager 32 warns the user when the signal strength of any battery pack radio 30 drops below some threshold. A different threshold and/or warning may be used for any battery pack 14 mated with a probe 12 and/or for a currently active or paired probe 12. Alternatively or additionally, the ultrasound imager 32 outputs that the battery pack 14 or any connected probe 12 will signal, and then causes the battery pack 14 or any connected probe 12 to output a visual or audio indication in order for the user to locate the battery pack 14 or probe. Other location actions may be performed in response to a measure of imager received signal or other location measure.

In another embodiment, the ultrasound imager 32 is configured to receive and output the charge or other battery status of the batteries 26. The status is received from any battery pack 14 using the integrated radio 30. The status for the battery pack 14 of the active probe 12 may be received. The status for the spare or other battery packs 14 may be received. The status of the spares may be treated or displayed differently than the status of a currently used or active battery pack 14 of a probe 12 being used. For example, the status for the active battery 26 may be updated more frequently or is provided automatically while the status of spares is updated less frequently and/or provided only upon request by the user or low charge in the active battery 26.

Since radios 30 are available for the various battery packs 30, the ultrasound imager 32 may receive or may request battery status from the active and spare battery packs 14 regardless of whether connected to a probe 12, charging station, or disconnected. The ultrasound imager 32 indicates to the user the charge states of all batteries in the vicinity. Other information may be indicated as well, such as whether the various battery packs 14 are mated to a probe 12. The ultrasound imager 32 displays an inventory of connected probes 12 and connected or not connected battery packs 14 in a vicinity (e.g., room) along with their associated charge and mating status.

Based on the inventory, the ultrasound imager 32 or user may determine which battery pack 14 to use or mate with a probe 12 for scanning. For example, the ultrasound imager 32 highlights the battery pack 14 with the most charge, such as by highlighting in a list on the display 34 and/or causing the battery pack 14 to output a visual and/or audio indicator. The ultrasound imager 32 or user may determine that all available batteries have too low of a charge, prompting a warning to charge. The battery packs 14 below a threshold charge level (e.g., 20%) may be identified so that the user may arrange for charging. Other charge or battery status related information may be used.

The radios 30 may be used by the ultrasound imager 32 to warn the user when a probe 12 has been mated with battery for a prescribed time interval. Alternatively or additionally, a life of the batteries 26 is tracked and the user is warned when a battery 26 is nearing a life expectancy. Any of various inventory control or tracking may be implemented using the battery and/or mating status information from the radios 30 of the battery packs 14. Location information may be included with the status output, such as indicating the mated batteries 26 and the un-mated battery packs 14 with sufficient charge that are closest to the ultrasound imager 32.

The locator 36 is a smartphone, tablet, personal computer, laptop, or specifically designed locator device. As a general-purpose device (e.g., smartphone or tablet), the locator 36 is configured by an application or program. As a specifically designed device, circuitry, design, and/or programming configured the locator 36 for use in the ultrasound system. The locator 36 is separate from or different from the ultrasound imager 32. The locator 36 is handheld or may be moved in one embodiment, allowing for locating battery packs 14 by change in signal strength and/or by triangulation.

The locator 36 includes a transceiver or receiver for communications with the radios 30 of the battery packs 14. The locator 36 may or may not also communicate wirelessly with the ultrasound imager 32.

Any of the various inventory, battery status, and/or location functions discussed above for the ultrasound imager 32 may be alternatively or additionally performed with the locator 36. The locator 36 may communicate with the radios 30 to gather information, request information, and/or control the battery packs 14 and/or connected probes 12. For example, location information (e.g., estimated distance or that the battery pack 14 has been triggered to signal) is output by the locator 36. The locator 36 may measure the signal strength or receive measured signal strength from the ultrasound imager 32.

Where the locator 36 is more mobile than the ultrasound imager 32, the locator 36 may more easily be used to locate battery packs 14 that are hidden or muffled (i.e., cannot see or hear signals from the battery pack 14) or that do not have an output. Instead, the locator 36 is moved to determine in which direction the signal strength increases or decreases. This information may be used to zero in or home in on the location of a specific battery pack 14. In one embodiment, an audible or visual indicator varies in proportion to the signal strength.

The locator 36 may output battery status or other status information for the battery packs 14. For example, the charge state and pairing is output for the various battery packs 14 in the vicinity or within range of the locator 36.

Any of the user interface functions may be provided on the locator 36. For example, the ultrasound imager 32 and/or probe 12 may be configured for scanning using inputs from the locator 36. In other embodiments, the locator 36 may receive an ultrasound image from the ultrasound imager 32 and display the images on the locator.

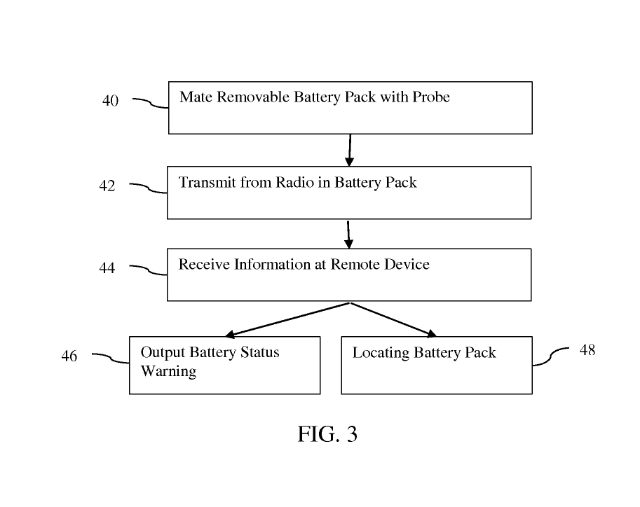

FIG. 3 is a flow chart of one embodiment of a method for communicating in an ultrasound system. The ultrasound system includes a wireless probe, a battery pack, a radio in the battery pack, and another device, such as an ultrasound imager or locator. The system of FIG. 1, FIG. 2, or a different system is used to implement the method. For example, a user performs the mating of act 40. The radio in the battery pack performs act 42. The locator or ultrasound imager performs acts 44, 46, and/or 48. Acts 46 and/or 48 may be performed, at least in part, by the battery pack or mated probe.

Additional, different, or fewer acts may be provided. For example, acts for requesting battery information are provided. As another example, other communications are provided, such as the radio in the battery pack receiving control instructions or communication requests. Some or all of the acts may be repeated for different probes, battery packs, locators, and/or ultrasound imagers. Acts for inventory control or charge control for a group of batteries may be provided. In yet another example, act 40, 46, and/or 48 are not performed.

The acts are performed in the order shown or different orders. For example, act 42 and/or act 44 are performed prior to act 40. Acts 46 and 48 may be performed simultaneously or in any order.

In act 40, a removable battery pack is mated with an ultrasound transducer probe. The pack connects physically and electronically with the probe. A latch, magnets, snap fit, screw, press fit, door, or other connection holds the pack to or in the probe. As part of this holding, electrical contact through any number of conductor pairs is established. The electrical contact provides for power from the pack to the probe. Control, measurement, and/or scan data may be exchanged over the same or other contacts, or by inductive coupling.

In alternative embodiments, the battery pack is not mated with a probe. The battery pack may be removed from the probe.

In act 42, the radio in the battery pack wirelessly transmits information. Using Bluetooth, Bluetooth low energy, other standard, or non-standard protocol, information is transmitted from the radio of the battery pack to one or more other devices. The broadcast may be addressed to a specific recipient or class of recipients or may not be addressed.

The information transmitted is battery status, mating status, or other status information. Alternatively or additionally, the radio in the battery pack transmits location information. The location information may be a response to a location signal request. The location information may be any signal without location information but which is used to determine signal strength by the remote device.

The radio in the battery pack, when mated, may transmit ultrasound data from the ultrasound transducer probe. Measures, signals, and/or data from the probe or sensors of the battery pack are formatted for wireless transmission and transmitted by the radio of the battery pack. The need for a working radio in the probe may be avoided by using the radio in the battery pack. In alternative embodiments, a radio or several radios in the probe outside of the battery pack transmits the ultrasound data or control data.

In act 44, the transmitted information is received at a remote device. A tablet, smartphone, ultrasound imager, or other remote device receives the information. A cable or wires do not connect the remote device to the battery pack or probe. The receipt is wireless, such as receiving the radio frequency transmission with a receiver or transceiver.

The received information is used for output to the user or response to the radio of the battery pack. For response, the remote device communicates back with the radio of the battery pack in order to request more information, confirm receipt, and/or control operation. For control, the control instructions, values, or settings are for the battery pack, radio, and/or connected probe. For example, the remote device determines that a signal strength of the transmission from the radio in the battery pack is below a threshold, so transmits back instructions to cease or prevent some operations and/or to output a visual or audio indicator for locating the specific battery pack. The user may follow the light or sound to identify or locate the battery pack.

For output to the user by the remote device, any output may be used. Status (e.g., charge or mating), location, ultrasound image, control settings, user interface, and/or other outputs are provided.

In act 46, the remote device outputs a battery status warning. A warning regarding low battery is output. The warning may be that none of the available or in range battery packs have sufficient (i.e., above a threshold level) charge. In alternative embodiments, the output is of a high charge, such as outputting an indication of the battery pack with the highest charge or outputting the level of the highest charge. The output may be inventory information, such as a list of battery packs and corresponding charge levels.

The output of the warning may be in conjunction with causing the battery pack to identify itself. The output is that the battery pack with the low charge, all of the battery packs with charge below a level, or the battery pack with the greatest charge will identify themselves with a noise or visual signal. The battery packs output the noise or visual signal at the same time or upon confirmation input by the user on the remote device.

In act 48, the received information is used for locating the battery pack. Location information may be output by the remote device. The signal strength or other information is used to indicate a location, such as a specific location or a distance. The output location information may be a warning that a battery pack is moving or is at a location beyond a threshold range from the remote device. The location may be a change in distance so that the holder of the remote device may home in on the battery pack. Alternatively or additionally, the battery pack output is activated.

The location information is provided regardless of mating. Spare battery packs may be located. Battery packs mated with probes may be located. Alternatively, the location output may or may not depend on the mating status. For example, the location information for an unmated probe is provide upon request, but location information for a mated probe is upon request or when moving away from the system by a given range. The location information allows a user to find misplaced batteries and/or probes.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A system for communications with an ultrasound scanner, the system comprising:
an ultrasound transducer probe comprising a probe housing and a transducer array in the probe housing;
a battery pack comprising a battery pack housing where the battery pack is configured for removable mating with the transducer probe; and
a wireless radio in the battery pack housing, the wireless radio configured for wireless communications with a remote device.

2. The system of claim 1 wherein the probe housing is sized and shaped for being held by a hand and is free of cable connection and wherein the transducer probe further comprises transmit and receive electronics within the probe housing.

3. The system of claim 2 wherein the battery pack, when mated with the transducer probe, powers the transmit and receive electronics.

4. The system of claim 1 wherein the battery pack further comprises a charge level sensor, and wherein the wireless radio is configured to transmit a charge level of the battery pack.

5. The system of claim 4 wherein the wireless radio is configured to transmit the charge level of the battery pack when the battery pack is not mated with the transducer probe.

6. The system of claim 1 wherein the probe housing includes a mating indention in which the battery pack housing fits so that part of the battery pack housing forms an outer surface with the probe housing.

7. The system of claim 1 wherein the wireless radio comprises a low-power radio.

8. The system of claim 1 wherein the wireless radio comprises a chip enclosed within the battery pack housing.

9. The system of claim 1 wherein the wireless radio is configured to transmit a locator signal at a predetermined amplitude.

10. The system of claim 1 wherein the wireless radio is configured to transmit ultrasound data from the transducer probe when the battery pack is mated with the transducer probe.

11. The system of claim 1 further comprising an additional wireless radio in the probe housing and outside of the battery pack housing, the additional wireless radio configured to transmit ultrasound data from the transducer probe, and wherein the wireless radio in the battery pack housing is configured to transmit a battery status.

12. An ultrasound system for communications, the ultrasound system comprising:
a battery enclosure enclosing a battery;
a cableless ultrasound transducer probe releasably connectable with the battery enclosure;
a transceiver within or on the battery enclosure; and
an ultrasound imager configured to generate an ultrasound image from data received from the cableless ultrasound transducer probe.

13. The ultrasound system of claim 12 wherein the battery powers a circuit of the cableless ultrasound transducer probe when the battery enclosure is connected with the cableless ultrasound transducer probe, and wherein the transceiver is configured to transmit the data when the battery enclosure is connected with the cableless ultrasound transducer probe.

14. The ultrasound system of claim 12 further comprising an additional transceiver configured to transmit the data from the cableless ultrasound transducer probe, and wherein the transceiver within or on the battery enclosure is configured to transmit a status of the battery regardless of whether the battery enclosure is connected with the cableless ultrasound transducer probe.

15. The ultrasound system of claim 12 wherein the transceiver is configured to transmit a signal and wherein the ultrasound imager or another unconnected device is configured to output location information in response to a measure of receipt of the signal.

16. The ultrasound system of claim 12 further comprising:
one or more additional battery enclosures enclosing respective batteries; and
one or more additional transceivers within or on respective battery enclosures, the transceiver and additional transceivers configured to transmit charge status of the respective batteries;
wherein the ultrasound imager or another unconnected device is configured to receive and output the charge status of the batteries.

17. The ultrasound system of claim 12 wherein the transceiver is configured to receive re-configuration instructions from the ultrasound imager.

18. A method for communicating in an ultrasound system, the method comprising:
mating a removable battery pack with an ultrasound transducer probe;
wirelessly transmitting information from a radio in the battery pack; and
receiving the information at a remote device.

19. The method of claim 18 wherein wirelessly transmitting the information comprises transmitting battery status, and
further comprising outputting by the remote device a warning when the battery status indicates a charge below a threshold.

20. The method of claim 18 further comprising locating the removable battery pack from the information when the removable battery pack is unmated with the ultrasound transducer probe.

21. The method of claim 18 wherein transmitting the information comprises transmitting ultrasound data from the ultrasound transducer probe, the transmitting being with the radio in the battery pack.
